(19) United States
(12) Patent Application Publication
(10) Pub. No.: US 2022/0218685 A1
(43) Pub. Date: Jul. 14, 2022

(54) COMPOSITION FOR TREATING EYE DISEASES

(71) Applicants: Samjin Pharmaceutical Co., Ltd., Seoul (KR); AptaBio Therapeutics Inc., Gyeonggi-do (KR)

(72) Inventors: Min-hyo Ki, Chungcheongnam-do (KR); Kyung-wan Nam, Gyeonggi-do (KR); Jin-cheul Kim, Seoul (KR); Kyung-jin Ahn, Seoul (KR); Sung Hwan Moon, Gyeonggi-do (KR); Soo Jin Lee, Gyeonggi-do (KR)

(73) Assignees: Samjin Pharmaceutical Co., Ltd., Seoul (KR); AptaBio Therapeutics Inc., Gyeonggi-do (KR)

(21) Appl. No.: 17/614,705

(22) PCT Filed: May 29, 2020
(86) PCT No.: PCT/KR2020/007047

§ 371 Date: Nov. 29, 2021

(30) Foreign Application Priority Data
May 31, 2019 (KR) 10-2019-0064486

(51) Int. Cl. A61K 31/4439; A61K 47/34; A61K 9/00; A61P 27/02

(52) U.S. Cl. CPC A61K 31/4439; A61P 27/02; A61K 9/0051; A61K 47/34

(57) ABSTRACT

The present invention relates to a composition for treating eye diseases, wherein the composition comprises a pyrazole-based compound, which is a pharmacologically active substance, and a biodegradable polymer, so that a drug effectively reaches a posterior segment of an eyeball, while prolonging an action time of the drug administered into the eyeball.

### TECHNICAL FIELD

The present invention relates to a pharmaceutical composition for treating eye diseases, comprising a pharmacologically active substance and a biodegradable polymer.

### BACKGROUND ART

An eye disease is a disease or disorder which affects or involves the eye or one of the parts or regions of the eye. The eye includes the eyeball and the tissues and fluid that make up the eyeball, the periocular muscles, and the portion of the optic nerve which is within or adjacent to the eyeball. Anterior segment ocular disease is a disease or disorder which affects or involves an anterior ocular region or area, such as a periocular muscle, an eyelid, or an eyeball tissues or fluids which is located anterior to the posterior wall of the lens capsule or ciliary muscles. In other words, an anterior segment ocular disease primarily affects or involves the conjunctiva, the cornea, the anterior chamber, the iris, the posterior chamber, the lens or the lens capsule and blood vessels and nerve which innervate an anterior ocular region or area. Posterior segment ocular disease is a disease or disorder which primarily affects or involves a posterior ocular region or area such as choroid or sclera, vitreous, vitreous chamber, retina, optic nerve, and blood vessels and nerve which innervate a posterior ocular region or area. Thus, posterior segment ocular diseases may include a disease or disorder, for example, macular degeneration (non-exudative age-related macular degeneration and exudative age-related macular degeneration, AMD); choroidal neovascularization; acute macular neuroretinopathy; macular edema (cystoid macular edema and diabetic macular edema); Behcet's disease, retinal disorders, diabetic retinopathy (including proliferative diabetic retinopathy); retinal arterial occlusive disease; retinal vein occlusion; uveitis; retinal detachment; ocular trauma which affects a posterior ocular area or location; a posterior segment ocular disease caused by or influenced by an ocular laser treatment; a posterior segment ocular diseases caused by or influenced by a photodynamic therapy, photocoagulation, radiation retinopathy, epiretinal membrane disorders, branch retinal vein occlusion, anterior ischemic optic neuropathy, non-retinopathy diabetic retinal dysfunction, retinitis pigmentosa, and glaucoma. Glaucoma can be considered a posterior segment ocular disease because the therapeutic goal is to prevent the loss of or reduce the occurrence of loss of vision due to damage to or loss of retinal cells or optic nerve cells (i.e. neuroprotection).

Angiogenesis refers to a process in which a new blood vessel sprouts from an existing microvessel, and the sprouting blood vessel proliferates to produce a new capillary blood vessel. Angiogenesis is a highly regulatory process that occurs in response to various pro-angiogenic stimuli such as growth factors, cytokines and other physiological molecules, as well as other factors such as hypoxia and low pH. Angiogenesis is a normal process which is very important in the development of embryos, fetal growth, placental proliferation, lutein formation, tissue regeneration and wound healing in the human body. However, there are diseases which angiogenesis itself can be the cause of a disease, if angiogenesis is abnormally increased or is not normally regulated.

Representative diseases associated with angiogenesis include eye diseases such as diabetic retinopathy, retinopathy of prematurity (ROP), age-related macular degeneration, corneal neovascularization, neovascular glaucoma, degeneration of spots, pterygium, retinitis pigmentosa, granular conjunctivitis and the like. In the eyeball, an angiogenic mechanism should be suppressed. However, if the angiogenic mechanism is activated due to a wrong signal, a serious eye disease may occur and thus become a major cause of acquired blindness. In particular, the retina is an organ that rapidly response to active oxygen due to its higher oxygen consumption than other muscles, and high concentration of glucose has been reported to promote a VEGF expression through activation of active oxygen, thereby inducing the progression and acceleration of eyeball damages. A therapy for eye diseases associated with angiogenesis includes laser treatment, photocoagulation, cryocoagulation, and photodynamic therapy. All of these therapy is a treatment by a surgery, and thus a drug therapy is still in a development stage. The treatment by a surgery has a great limitation in that it cannot be applied to all patients, and the success rate is low. Further, the treatment requires such a high cost as to lay a social and financial burden.

The eyeball is comprised of an anterior segment and a posterior segment. Due to such a various structure, each of tissues plays a role in interfering with drug delivery, thus making it very difficult to deliver a drug into the eyeball. A commonly used method for delivering a drug into the eyeball includes systemic circulation after oral administration, dripping of eye drops, intraocular injection and the like, but all of the three methods have a disadvantage of being limited. First of all, even if the drug is administered for the purpose of systemic circulation, it is known that it is very difficult for the drug to migrate into the eyeball due to the anatomic features inside the enclosed eyeball, which allows a very limited blood circulation under the influence of a blood-aqueous barrier, a blood-retinal barrier and the like. The method of dropping a drug by using eye drops is most commonly used now, but is known to have a very low bioavailability, in that only about 20% or less of the drug migrates into the cornea due to reflux blinking and so on after administration, and only about 5% thereof is delivered into the eyeball tissues (Schoenwald R D, Clin. Pharmacokinet. 1990 April; 18(4):255-69, Gaudana R, et al., Pharm. Res. 2009 May; 26(5):1197-216). In addition, the method of using eye drops has a problem in that it is difficult to deliver the drug to the posterior segment of the eye due to a complicated structure inside the eyeball.

In order to solve the above problems, a method of directly injecting a drug into a vitreous body (intravitreal injection) has been used to deliver the drug into the eyeball. However, this therapy has a limit in a single dosage, resulting in the poor patient compliance with repeated injection. This repeated process has an increasing possibility to cause side effects such as retinal detachment, endophthalmitis and cataract (Evaluation of the retinal toxicity and pharmacokinetics of dexamethasone after intravitreal injection, Arch. Ophthalmol. 110:259-66).

In result, there is a need to develop a drug delivery system which effectively acts on the posterior segment of the eyeball and also prolongs the action time of the drug administered into the eyeball in order to reduce the number of intraocular injections.

### PRIOR ART REFERENCES

### Patent Documents

(Patent Document 1) Korean Patent Registration No. KR 10-1821593

### Non-Patent Documents

(Non-Patent Document 1) Pharm. Res. 2009 May; 26(5):1197-216
(Non-Patent Document 2) Arch. Ophthalmol. 110:259-66

### DISCLOSURE OF INVENTION

### Technical Problem

An objective of the present invention is to provide a composition for treating eye diseases, which comprises a pyrazole-based compound and a biodegradable polymer, and in which a drug effectively reaches a posterior segment of an eyeball, while prolonging the action time of the drug administered into the eyeball.

### Solution to Problem

This is described in detail as follows. Meanwhile, each description and embodiment disclosed herein may be also applied to other descriptions and embodiments thereof, respectively. In other words, all the combinations of various elements disclosed herein fall within the scope of the present invention. Also, it may not be seen that the scope of the present invention is limited to the specific descriptions described below.

The present invention provides a pharmaceutical composition for treating eye diseases, comprising a pyrazole-based compound represented by a following chemical formula 1 or pharmaceutically acceptable salts thereof; and a biodegradable polymer:

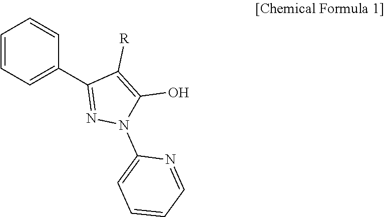

(wherein R is straight or branched alkyl group having 1 to 10 carbon atoms.)

In one embodiment of the present invention, the pyrazole-based compound may be 3-phenyl-4-propyl-1-(pyridine-2-yl)-1H-pyrazole-5-ol or pharmaceutically acceptable salts thereof, and the salt may be hydrochloride.

In the present invention, the “biodegradable polymer” means a polymer in which a product degraded by hydrolysis, enzyme reaction, or various mechanisms occurred in vivo has bio-compatibility or is non-toxic. The biodegradable polymer may be degraded into a non-toxic substance after releasing a drug, which can be naturally removed in the metabolic process. The polymer is slowly degraded into small pieces so as to play a role in releasing the drug. Thus, the polymer is very useful in drug delivery.

In the present invention, if a degraded result is a physiologically acceptable degradation product, the biodegradable polymer that can be used include a polymer which is comprised of a monomer such as organic ester or ether, but is not limited thereto. The polymer is generally a condensation polymer, which may be cross-linked or non-cross-linked. In case of cross-linking, there was a low degree of cross-linking, in which linkage is made less than 5%, cross-linkage is generally made less than 1%. In most cases, the polymer may contain oxygen and nitrogen, in particular, oxygen, besides carbon and hydrogen. Oxygen may be present as oxy, for example hydroxyl or ether, carbonyl, for example non-oxo-carbonyl such as carboxylic acid ester, or the like. Nitrogen may be present as amide, cyano and amino. Hydroxy aliphatic carboxylic acid, homo- or co-polymer and polysaccharide may be important in particular. Among the important polyester are D-lactic acid, L-lactic acid, racemic lactic acid, glycolic acid, caprolactone, and a homo- or co-polymer of a mixture thereof. A co-polymer of glycolic acid and lactic acid, or lactic acid is important in particular, and a biodegradation rate may be adjusted depending on a ratio between glycolic acid and lactic acid. A ratio between glycolic acid and lactic acid, which are respective monomers among poly(lactic-co-glycolic) acid (PLGA) co-polymer, may be 0:100 to about 50:50, specifically, 0:100, about 15:85, about 25:75, about 35:65, or about 50:50. In this case, polylactic acid (PLA) corresponds to the case, in which a ratio between glycolic acid and lactic acid, which are respective monomers out of poly(lactic-co-glycolic) acid (PLGA) co-polymer, is 0:100.

A specific example of the biodegradable polymer may be selected from the group consisting of collagen, chitosan, poly(propylene fumarate), poly(lactic-co-glycolic)acid (PLGA), polylactic acid (PLA), polyglycolic acid (PGA), polycaprolactone (PCL), lactide/caprolactone copolymer (PLC), poly(L-lactide) (PLLA) and a mixture thereof, but is not limited thereto. Any material may be allowed as long as they conform to the definition of the biodegradable polymer and enables a delayed release of a pyrazole-based compound. As one example, the biodegradable polymer may be poly(lactic-co-glycolic)acid (PLGA), poly(lactic) acid (PLA), or a mixture thereof.

Particularly, in the present invention, the biodegradable polymer may use a polymer comprising a hydrophilic and hydrophobic ended PLA or PLGA, which is useful to modulate polymer degradation rates. The hydrophobic ended (also referred to as capped or end-capped) PLA and PLGA have a hydrophobic ester linkage at the polymer terminus. Typical hydrophobic end groups include alkyl esters and aromatic esters, but is not limited thereto. Hydrophilic ended (also referred to as uncapped) PLA and PLGA have a hydrophilic end group at the polymer terminus. PLA and PLGA with a hydrophilic end groups at the polymer terminus degraded faster than hydrophobic ended PLA and PLGA because it takes up water and undergoes hydrolysis at a faster rate. Examples of suitable hydrophilic end groups that may be incorporated to enhance hydrolysis include carboxyl, hydroxyl and polyethylene glycol, but is not limited thereto. A specific end group will typically vary depending on the initiator employed in a polymerization process.

In the present invention, the biodegradable polymer may contain a pyrazole-based compound dispersed therein, and the pyrazol-based compound can be homogeneously dispersed in the biodegradable polymer. The selection of the biodegradable polymer used can vary with the purposes, and the desired release kinetics, the nature of the disease to be treated, etc. Polymer characteristics that are considered include the biocompatibility and biodegradability at the ocular area to be implanted, as well as compatibility with the active agent, processing temperatures, and the like.

In the present invention, the biodegradable polymer may be contained in an amount of 10 to 90% by weight with regard to the total weight of the composition. A pharmaceutical composition of the present invention may contain the biodegradable polymer in the range of by weight above, thereby delaying a time for which a drug bound with the biodegradable polymer is released into the free form.

In the present invention, the pyrazole-based compound represented by the above chemical formula 1 or pharmaceutically acceptable salts thereof may be contained in an amount of 10 to 90% by weight with regard to the total weight of the composition.

Specifically, the composition may release a pyrazole-based compound represented by the chemical formula 1 or pharmaceutically acceptable salts thereof, which are contained in the composition, in an amount of about 50% by weight or less for four weeks after starting a release test, thereby showing an effect of prolonging the action time of the drug. The release test is performed by using a method of United States Pharmacopeia (USP) Dissolution Apparatus 3 (Reciprocating Cylinder). Such prolonged effect of drug release may be adjusted by considering factors including a type of eye disease, severity, activity of the drug, sensitivity to the drug, a treatment period and a concurrently used drug, as well as other factors well known in a medical field.

In the present invention, the composition may be parenterally administered, and specifically may be administered into an ocular area.

In the present invention, the “eyeball” is a spherical structure located in the orbit of a skull and is an organ responsible for vision. The “ocular area” means an inside of the eyeball of the individual, an outside thereof, or an area adjacent thereto. Specifically, the ocular area may be sclera (intrascleral), episclera (transscleral), vitreous cavity, choroid, cornea, stroma, intracameral, aqueous humor, lens, fornix or optic nerves, and more specifically the vitreous cavity.

With regard to the administration, the composition may be parenterally administered into mammals including humans, particularly the ocular area, and more particularly vitreous cavity. Specifically, the composition may be administered according to various methods including cutting a sclera followed by inserting the composition therein by using forceps, a trocar or other types of applicator. In some examples, the trocar or the applicator may be used without cutting. An administration method may include accessing a target region of the ocular area with a needle, which means once entering the target region, that is, the vitreous cavity, but is not limited thereto.

The composition of the present invention may be prepared into an implant formulation.

In the present invention, the “implant” is meant to include an ocular implant or a drug delivery device, which may be inserted into any position of the eyeball, while releasing a controlled amount of an active ingredient over a prolonged time including several days, several weeks or several months. The implant may be biocompatible and are formed from biodegradable materials such as biodegradable polymers.

The implant has highly homogeneous characteristic, and thus may deliver a precise and accurate dosage of an active ingredient continuously, providing a release of the active ingredient at a highly controlled rate within the eyeball over a certain time period. The active ingredient released from the implant of the present invention may selectively target a certain region of the eyeball. For example, the active ingredient may be released from the implant located in a patient's posterior segment, providing a therapeutic advantage to the retina of eye or a part of it.

Factors which influence the release kinetics of the implant formulation may include characteristics such as a particle size of an active agent, the solubility of the active agent, the ratio of active agent to polymer, the method of manufacture, the surface area of the implant, and the erosion rate of the polymer. A biodegradable implants are generally solid, and may be formed as particles, sheet patches, films, discs, rods (cylindrical shape), etc., but may be of any size or shape compatible with the selected site of implantation, as long as the implants have the targeted release kinetics and deliver an effective amount of active agent for the intended eye disease. Tolerance for the implant at the site of implantation will be determined by factors such as size limitation on insertion, ease of handling, and a patient's compliance. The vitreous chamber is able to accommodate relatively large rod-shaped implants, generally having diameters of about 0.05 to 3.0 mm and a length of 0.5 to 10.0 mm. In one example, the “rod shape” may be a cylindrical rod shape of which cross section is substantially a circle. The rod-shaped implants provide an advantage of being easily transplanted and not being accompanied by discomfort thereafter. Preferably, the rods may have diameters of about 1.0 to 2.0 mm, but is not limited thereto. It may be preferable to use the implant which has approximately similar volumes, though a shape thereof having variable geometries.

Specifically, in the present invention, a length of the implants may be 0.5 to 10 mm. Considering that a human eyeball size is generally 24 mm, it may be appropriate to administer the implant into the eyeball, if the length of the implant is 10 mm or less. However, if the length of the implant is more than 10 mm, there may be a problem in that the implant is easily broken when being inserted by using an applicator or a catheter. Also, if the length of the implant is less than 0.5 mm, there may be a disadvantage in that it is difficult to handle the implant due to an inconvenient charge of the applicator or the catheter.

Eye diseases which may be prevented or treated by the pharmaceutical composition according to the present invention include the eye diseases which may show all characteristic of anterior segment ocular diseases, posterior segment ocular diseases, or anterior segment ocular diseases and posterior segment ocular diseases. Specifically, the eye diseases may include diabetic retinopathy (DR), diabetic macular edema, age-related macular degeneration, retinopathy of prematurity (ROP), polypoidal choroidal vasculopathy, ischemic proliferative retinopathy, retinitis pigmentosa, cone dystrophy, proliferative vitreoretinopathy (PVR), retinal arterial occlusion, retinal vein occlusion, pterygium, retinitis, corneitis, conjunctivitis, uveitis, Leber's hereditary optic neuropathy, retinal detachment, retinal pigment epithelial detachment, neovascular glaucoma, corneal neovascularization, retinal neovascularization, choroidal neovascularization (CNV), eye diseases caused by viral infection, and the like. Preferably, the eye diseases may include the eye diseases selected from the group consisting of diabetic retinopathy (DR), age-related macular degeneration, retinitis, corneitis, conjunctivitis, uveitis, corneal neovascularization, retinal neovascularization and choroidal neovascularization (CNV).

When preparing the composition of the present invention, other excipients may be further used for various purposes. For example, buffering agents, anti-oxidants, preservatives and the like may be used. An example of buffering agents which can be used may include sodium carbonate, sodium bicarbonate, sodium phosphate, sodium borate, sodium acetate, sodium chloride, potassium chloride and the like, but is not limited thereto. An example of anti-oxidants which can be used may include butyl hydroxy toluene, butyl hydroxy anisole, sodium sulfite, sodium bisulfite, sodium metabisulfite, ascorbic acid, cysteine hydrochloride, cystine, thioctic acid, thioglycerol and the like, but is not limited thereto. An example of preservatives which can be used may include sodium bisulfate, sodium bisulfite, sodium trisulfate, benzalkonium chloride, chlorobutanol, thimerosal, phenylmercury acetate, methylparaben, propylparaben and phenylethyl alcohol, but is not limited thereto. Also, an additional hydrophilic or hydrophobic compound, which promotes or delays a release of an active agent, may be included.

A method for preparing the pharmaceutical composition may use an extrusion molding method, which enables a large-scale production and makes it possible to obtain an implant in which the drug is homogeneously dispersed in the polymer. In case of using the extrusion molding method, the method may be performed at a temperature of about 25 to 150° C., preferably 60 to 130° C.

A dosage of the composition may be administered in an amount of 300 ng to 100 mg, and may be appropriately selected by those skilled in the art depending on a patient's state and weight, a degree of disease, a type of drug, an administration time, etc.

The pharmaceutical composition according to the present invention may be administered in a pharmaceutically effective amount. In the present invention, the “pharmaceutically effective amount” means an amount sufficient to treat the disease at a reasonable benefit/risk ratio applicable to medical treatment, and a level of effective dose may be determined by factors including a patient's disease type, severity, activity of a drug, sensitivity to the drug, an administration time, an administration route and excretion rate, a treatment period and a concurrently used drug, as well as other factors well known in a medical field.

The pharmaceutical composition of the present invention may be administered as an individual therapeutic agent or in combination with other therapeutic agents, may be administered sequentially or simultaneously with a conventional therapeutic agent, and may be administered in a single or multiple doses. Considering all of the above factors, it is important to administer an amount that can obtain the maximum effect in a minimum amount without a side effect, and the amount may be easily determined by those skilled in the art.

The present invention also includes a method of administering the pharmaceutical composition of the present invention into an ocular area of humans or mammals excluding humans. In general, the present invention includes administering for example, injecting or disposing, the pharmaceutical composition of the present invention into the ocular area, for example, a posterior segment, for example, vitreous cavity, of humans or mammals excluding humans. Such administration step is effective to provide the desired therapeutic effect to the tissues of the posterior segment. Preferably, the administration step may include at least one of injection or disposition into vitreous cavity, subconjunctival injection or disposition, sub-tenon injection or disposition, retrobulbar injection or disposition, suprachoroidal injection or disposition, etc.

The pharmaceutical composition of the present invention may be disposed in the posterior segment through a 18 to 22 gauge needle so as to be administered into an inside of the eye, for example, vitreous cavity.

The present invention also provide a method for treating eye diseases, comprising administering the pharmaceutical composition of the present invention into the ocular area, for example, the posterior segment, of humans or mammals excluding humans.

Specifically, an effective amount of the compound according to the present invention may vary depending on the age, sex, and weight of the patient, and may be administered daily or every other day or may be administered once a month, once every three months, or once every six to twelve months.

The pharmaceutical composition of the present invention may release a pyrazole-based compound represented by the chemical formula 1 or pharmaceutically acceptable salts thereof, which are contained in the composition, in an amount of about 50% by weight or less for four weeks after starting a release test, when carrying out a release test by using a method of United States Pharmacopeia (USP) Dissolution Apparatus 3 (Reciprocating Cylinder). Also, the pharmaceutical composition of the present invention may release the pyrazole-based compound represented by the chemical formula 1 or pharmaceutically acceptable salts thereof, which are contained in the composition, in an amount of about 70% by weight or less for eight weeks when carrying out a release test by using the method of United States Pharmacopeia (USP) Dissolution Apparatus 3 (Reciprocating Cylinder).

The pharmaceutical composition of the present invention may release the pyrazole-based compound represented by the above chemical formula 1 or pharmaceutically acceptable salts thereof at a rate of about 7 to about 180 ug/week.

In one experimental example of the present invention, as a result of measuring an in vitro drug release of the composition containing a biodegradable polymer, it was identified that the drug release is prolonged in an amount of about 50% by weight or less at 4 weeks and about 70% by weight or less at 8 weeks after starting a release test by using the method of United States Pharmacopeia (USP) Dissolution Apparatus 3 (Reciprocating Cylinder) (see Experimental Example 2).

Also, in another experimental example of the present invention, as a result of evaluating the in vivo pharmacokinetics of the composition containing the biodegradable polymer, it was identified that the drug release is prolonged to increase bioavailability, thereby confirming that the composition of the present invention has an excellent sustained release of the drug within the vitreous body (see Experimental Example 3).

In other words, the pharmaceutical composition of the present invention has an effect of prolonging an action time of the drug administered into the eyeball since the pyrazole-based compound exhibiting a valid effect on treatment of eye diseases is entrapped in the biodegradable polymer. Thus, such pharmaceutical composition may make it easy to deliver the drug into the eyeball without a risk of residual degradation products therein and may also prevent side effects such as retinal detachment, endophthalmitis, etc., which are caused by repeated administration through injection.

### Advantageous Effects of Invention

A composition of the present invention comprises a pyrazole-based compound, which exhibits a valid effect on treating eye diseases, and a biodegradable polymer. It is excellent in drug migration into an inside of an eyeball since the drug effectively reaches a posterior segment of an eyeball, while exhibiting an effect of prolonging an action time of the drug administered into the eyeball.

### BRIEF DESCRIPTION OF DRAWINGS

FIG. 1 is a graph showing the results of in vitro cumulative release rate of a drug.

FIG. 2 is a graph showing the effect of in vivo delayed release of a drug.

### MODE FOR THE INVENTION

Hereinafter, the present invention will be described in more detail through examples. These example are provided only for the purpose of illustrating the present invention in more detail, and thus it will be apparent to those skilled in the art that the scope of the present invention is not limited thereto according to the principles of the present invention.

### Examples 1 to 3. Preparation of Implant Containing Pyrazole-Based Compound

APX-115 (3-phenyl-4-propyl-1-(pyridine-2-yl)-1H-pyrazole-5-ol hydrochloride), which is a pyrazole-based compound, and PLA 203H (Resomer® R 203H, Evonik, Germany) were accurately weighed as shown in Table 1 below, and placed in a stainless steel mixing container. The container was sealed, then placed in a tubular mixer, and then mixed at 96 rpm for 20 minutes. The obtained mixed powder was fed into a hot-melt extruder (Process-11, Thermo, USA), and set to predetermined temperature 85° C. and 12 rpm of a screw speed. A filament was subjected to extrusion molding according to a guide mechanism, and an implant was cut into a cylindrical rod shape having a diameter of 0.5 to 0.6 mm and containing 720 ug of an active agent.

TABLE 1

| | Example 1 | Example 2 | Example 3 |
|---|---|---|---|
| Pyrazole-based compound (g) | 20.0 | 20.0 | 18.0 |
| Resomer ® R 203H (g) | 20.0 | 10.0 | 2.0 |
| Weight ratio of drug:polymer (w/w) | 1:1 | 2:1 | 9:1 |
| Weight ratio of polymer in implant | 50.0% | 33.3% | 10.0% |
| (%) | | | |

### Examples 4 to 6. Preparation of Implant Containing Pyrazole-Based Compound

APX-115 (3-phenyl-4-propyl-1-(pyridine-2-yl)-1H-pyrazole-5-ol hydrochloride), which is a pyrazole-based compound, and PLA 203H (Resomer® R 203H, Evonik, Germany) were accurately weighed as shown in Table 2 below, and an implant was prepared by using the same preparation method as shown in Example 1 above. A filament was subjected to extrusion molding according to a guide mechanism, and the implant was cut into a cylindrical rod shape having a diameter of 0.5 to 0.6 mm and containing 180 ug of an active agent.

TABLE 2

| | Example 4 | Example 5 | Example 6 |
|---|---|---|---|
| Pyrazole-based compound (g) | 20.0 | 10.0 | 10.0 |
| Resomer ® R 203H (g) | 40.0 | 40.0 | 90.0 |
| Weight ratio of drug:polymer (w/w) | 1:2 | 1:4 | 1:9 |
| Weight ratio of polymer in implant | 66.7% | 80.0% | 90.0% |
| (%) | | | |

### Examples 7 to 21. Preparation of Implant Containing Pyrazole-Based Compound

APX-115 (3-phenyl-4-propyl-1-(pyridine-2-yl)-1H-pyrazole-5-ol hydrochloride), which is a pyrazole-based compound, and a polymer of Table 3 below (Resomer®, Evonik, Germany) were used to prepare an implant according to the same preparation method as shown in Example 1 above. A filament was subjected to extrusion molding according to a guide mechanism, and the implant was cut into a cylindrical rod shape having a diameter of 0.5 to 0.6 mm and containing 720 ug of an active agent.

TABLE 3

| | Example 7 | Example 8 | Example 9 | Example 10 | Example 11 |
|---|---|---|---|---|---|
| Pyrazole-based compound (g) | 20.0 | 20.0 | 20.0 | 20.0 | 20.0 |
| Resomer ® RG 502 (g) | 20.0 | — | — | — | — |
| Resomer ® RG 502H (g) | — | 20.0 | — | — | — |
| Resomer ® RG 503 (g) | — | — | 20.0 | — | — |
| Resomer ® RG 504H (g) | — | — | — | 20.0 | — |
| Resomer ® RG 505 (g) | — | — | — | — | 20.0 |
| | Example 12 | Example 13 | Example 14 | Example 15 | Example 16 |
| Pyrazole-based compound (g) | 20.0 | 20.0 | 20.0 | 20.0 | 20.0 |
| Resomer ® RG 752H (g) | 20.0 | — | — | — | — |
| Resomer ® RG 753H (g) | — | 20.0 | — | — | — |
| Resomer ® RG 753S (g) | — | — | 20.0 | — | — |
| Resomer ® RG 756S (g) | — | — | — | 20.0 | — |
| Resomer ® RG 858S (g) | — | — | — | — | 20.0 |
| | Example 17 | Example 18 | Example 19 | Example 20 | Example 21 |
| Pyrazole-based compound (g) | 20.0 | 20.0 | 20.0 | 20.0 | 20.0 |
| Resomer ® RG 653H (g) | 20.0 | — | — | — | — |
| Resomer ® R 202S (g) | — | 20.0 | — | — | — |
| Resomer ® R 202H (g) | — | — | 20.0 | — | — |
| Resomer ® R 203S (g) | — | — | — | 20.0 | — |
| Resomer ® R 205S (g) | — | — | — | — | 20.0 |

### Comparative Examples 1 to 3. Preparation of Implant Containing Pyrazole-Based Compound

APX-115 (3-phenyl-4-propyl-1-(pyridine-2-yl)-1H-pyrazole-5-ol hydrochloride), which is a pyrazole-based compound, and PLA 203H (Resomer® R 203H, Evonik, Germany) were accurately weighed as shown in Table 4 below, and an implant was prepared by using the same preparation method as shown in Example 1. A filament was subjected to extrusion molding according to a guide mechanism, and the implant was cut into a cylindrical rod shape having a diameter of 0.5 to 0.6 mm and containing 720 ug of an active agent.

TABLE 4

| | Comparative | Comparative | Comparative |
|---|---|---|---|
| | Example 1 | Example 2 | Example 3 |
| Pyrazole-based compound (g) | 20.0 | 20.0 | 20.0 |
| Resomer ® R 203H (g) | 1.0 | 0.67 | 0 |
| Weight ratio of | 20:1 | 30:1 | 1:40 |
| drug:polymer (w/w) | | | |
| Weight ratio of polymer in | 4.8% | 3.2% | 0.0% |
| implant (%) | | | |

### Comparative Examples 4 to 6. Preparation of Implant Containing Pyrazole-Based Compound

APX-115 (3-phenyl-4-propyl-1-(pyridine-2-yl)-1H-pyrazole-5-ol hydrochloride), which is a pyrazole-based compound, and PLA 203H (Resomer® R 203H, Evonik, Germany) were accurately weighed as shown in Table 5 below, and an implant was prepared by using the same preparation method as shown in Example 1 above. A filament was subjected to extrusion molding according to a guide mechanism, and the implant was cut into a cylindrical rod shape having a diameter of 0.5 to 0.6 mm and containing 180 ug of an active agent.

TABLE 5

| | Comparative | Comparative | Comparative |
|---|---|---|---|
| | Example 4 | Example 5 | Example 6 |
| Pyrazole-based compound (g) | 2.0 | 2.0 | 2.0 |
| Resomer ® R 203H (g) | 40.0 | 60.0 | 80.0 |
| Weight ratio of | 1:20 | 1:30 | 1:40 |
| drug:polymer (w/w) | | | |
| Weight ratio of polymer in | 95.2% | 96.8% | 97.6% |
| implant (%) | | | |

### Experimental Example 1. Identification of Physical Properties

A filament was subjected to extrusion molding according to a guide mechanism. Diameters and lengths were measured using vernier calipers to compare and evaluate whether the prepared Examples 1, 2 and 6 and Comparative Examples 1 to 4 have an appropriate size for ocular administration and the results are shown in Table 6 below.

TABLE 6

| | | | | Comparative | Comparative | Comparative | Comparative |
|---|---|---|---|---|---|---|---|
| | Example 1 | Example 2 | Example 6 | Example 1 | Example 3 | Example 4 | Example 6 |
| Weight ratio of | 1:1 | 2:1 | 1:9 | 10:1 | 20:1 | 1:20 | 1:40 |
| drug:polymer (w/w) | | | | | | | |
| Diameter (mm) | 0.55 | 0.54 | 0.56 | 0.57 | 0.53 | 0.55 | 0.54 |
| Length (mm) | 5.02 | 3.81 | 9.62 | 3.13 | 2.83 | 12.67 | 22.51 |

As identified in the Table 6 above, given that an average human eyeball has a size of 24 mm, in the case of Examples 1, 2 and 6 and Comparative Examples 1 and 3, it was confirmed that the length was 10 mm or less and was suitable for ocular administration. On the other hand, in the case of Comparative Examples 4 and 6, it was confirmed that the length was more than 10 mm and was not suitable for intraocular administration, and it was not suitable as an implant for ocular administration because it is easily broken when is inserted using an applicator or catheter.

### Experimental Example 2. In Vitro Release Test

An in vitro release profile was measured by using a method of United States Pharmacopeia (USP) Dissolution Apparatus 3 (Reciprocating Cylinder). An implant sample, of which weight was measured, was administered into phosphate buffer saline (PBS, pH 7.4) solution maintained at 37° C. so as to measure a release rate of an active agent (APX-115). In this case, the volume of the solution is the volume at which the concentration of the active agent after release becomes 20% or less of the saturated state. The test was performed for 12 weeks and the concentration of the active agent was measured by using the HPLC.

<Analysis Conditions>
Column: Agilent C18 (250×4.6 mm, 5 μm)
Column temperature: 25° C.
Mobile phase: 0.1% Formic acid:Acetonitrile=20:80 (v/v)
Flow rate: 1.0 mL/min
Injection amount: 10 μL
Wavelength: 293 nm
Analysis time: 20 minutes

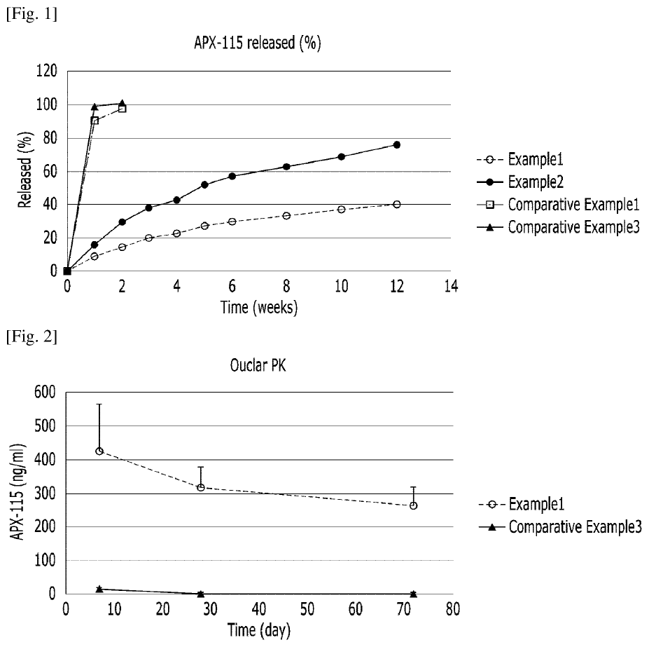

As a result of test on identifying an in vitro drug release amount of APX-115, as shown in FIG. 1, in Examples 1 and 2 having a high ratio of the polymer in the implant, accumulative release rate of APX-115 exhibited 50% by weight or less at 4 weeks and 70% by weight or less at 8 weeks after starting the release test, thus it was identified that there is an effect of prolonging the release. On the other hand, if Comparative Examples 1 and 3 were 90% or more released in one week, thereby if the ratio of the polymer in the implant is low, it was identified that the ratio was insufficient to form a delayed release implant.

### Experimental Example 3. Animal Test

New Zealand White rabbits having a weight of 2.0 to 2.7 kg were prepared and divided into two group: a test group and a control group. A preparation of the Example 1 was used for the test group, while a preparation of the Comparative Example 3 was used for the control group. A conjunctiva and a sclera between 10 o'clock and 12 o'clock were incised with a blade, and an implant was implanted into the posterior segment of a rabbit's right eye. After implantation with the preparation of Example 1 and the preparation of Comparative Example 3, an eyeball was removed at 7, 28 and 72 days after so as to identify a release amount of APX-115 within a vitreous body. The test was conducted on the test group and the control group by using respective three rabbits for each date, and the vitreous body was extracted from the removed eyeball and stored at −70° C. until an analysis was performed. A concentration of a main drug component in the vitreous body was analyzed by using LC-MS/MS so as to evaluate the pharmacokinetics (PK) in the rabbit's vitreous body.

As a result of test, as shown in FIG. 2, it was identified that Comparative Example 3 (control group) formed of an active agent alone without a biodegradable polymer shows a very fast loss of APX-115 within the vitreous body, while in Example 1 (test group), APX-115 was delayed to release, thereby increasing bioavailability. Thus, it was confirm that the composition of the present invention has an excellent sustained release of the drug within the vitreous body.

1. A pharmaceutical composition for treating eye diseases, comprising a pyrazole-based compound represented by a following chemical formula 1 or pharmaceutically acceptable salts thereof; and a biodegradable polymer. 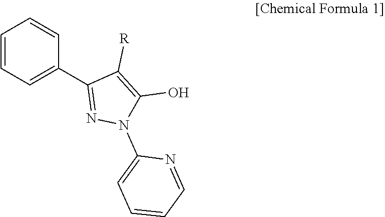
(wherein R is straight or branched alkyl group having 1 to 10 carbon atoms.)

2. The pharmaceutical composition for treating eye diseases according to claim 1, wherein the pyrazole-based compound is 3-phenyl-4-propyl-1-(pyridine-2-yl)-1H-pyrazole-5-ol or pharmaceutically acceptable salts thereof.

3. The pharmaceutical composition for treating eye diseases according to claim 2, wherein the pharmaceutically acceptable salt is hydrochloride.

4. The pharmaceutical composition for treating eye diseases according to claim 1, wherein the biodegradable polymer is selected from the group consisting of collagen, chitosan, poly(propylene fumarate), poly(lactic-co-glycolic)acid (PLGA), polylactic acid (PLA), polyglycolic acid (PGA), polycaprolactone (PCL), lactide/caprolactone copolymer (PLC), poly(L-lactide) (PLLA) and a mixture thereof.

5. The pharmaceutical composition for treating eye diseases according to claim 1, wherein the biodegradable polymer is poly(lactic-co-glycolic)acid (PLGA), poly(lactic) acid (PLA) or a mixture thereof.

6. The pharmaceutical composition for treating eye diseases according to claim 1, wherein the biodegradable polymer is comprised in an amount of 10 to 90% by weight with regard to the total weight of the composition.

7. The pharmaceutical composition for treating eye diseases according to claim 1, wherein the composition releases the pyrazole-based compound represented by the chemical formula 1 or pharmaceutically acceptable salts thereof, contained in the composition, in an amount of 50% by weight or less for 4 weeks after starting a release test.

8. The pharmaceutical composition for treating eye diseases according to claim 7, wherein the release test uses a method of United States Pharmacopeia (USP) Dissolution Apparatus 3 (Reciprocating Cylinder).

9. The pharmaceutical composition for treating eye diseases according to claim 1, wherein the composition is administered into an ocular area.

10. The pharmaceutical composition for treating eye diseases according to claim 9, wherein the ocular area is sclera (intrascleral), episclera (transscleral), vitreous cavity, choroid, cornea, stroma, intracameral, aqueous humor, lens, fornix or optic nerves.

11. The pharmaceutical composition for treating eye diseases according to claim 9, wherein the ocular area is the vitreous cavity.

12. The pharmaceutical composition for treating eye diseases according to claim 1, wherein the composition is an implant formulation.

13. The pharmaceutical composition for treating eye diseases according to claim 12, wherein the implant formulation is a rod-shaped implant and a length of the rod-shaped implant is 0.5 to 10 mm.

14. The pharmaceutical composition for treating eye diseases according to claim 12, wherein the implant formulation is the rod-shaped implant and a diameter of the rod-shaped implant is 0.1 to 2.0 mm.

15. The pharmaceutical composition for treating eye diseases according to claim 1, wherein the eye diseases are selected from the group consisting of diabetic retinopathy (DR), diabetic macular edema, age-related macular degeneration, retinopathy of prematurity (ROP), polypoidal choroidal vasculopathy, ischemic proliferative retinopathy, retinitis pigmentosa, cone dystrophy, proliferative vitreoretinopathy (PVR), retinal arterial occlusion, retinal vein occlusion, pterygium, retinitis, corneitis, conjunctivitis, uveitis, Leber's hereditary optic neuropathy, retinal detachment, retinal pigment epithelial detachment, neovascular glaucoma, corneal neovascularization, retinal neovascularization and choroidal neovascularization (CNV).

16. A method for treating eye diseases, comprising administering a pharmaceutical composition comprising a pyrazole-based compound represented by a following chemical formula 1 or pharmaceutically acceptable salts thereof; and a biodegradable polymer into humans or mammals excluding the humans. 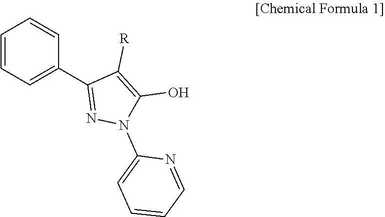
(wherein, R is straight or branched alkyl group having 1 to 10 carbon atoms.)
